(19) United States
(12) Patent Application Publication
(10) Pub. No.: US 2025/0356984 A1
(43) Pub. Date: Nov. 20, 2025

(54) MANAGEMENT OF MEDICATION DELIVERY FAILURES

(71) Applicant: CAREFUSION 303, INC., San Diego, CA (US)

(72) Inventor: Daniel M. ABAL, San Diego, CA (US)

(21) Appl. No.: 19/106,613

(22) PCT Filed: Aug. 29, 2023
(86) PCT No.: PCT/US2023/031441

§ 371 Date: Feb. 25, 2025

Related U.S. Application Data
(60) Provisional application No. 63/401,977, filed on Aug. 29, 2022.

(51) Int. Cl. G16H 20/17

(52) U.S. Cl. CPC G16H 20/17

(57) ABSTRACT

The disclosed device, system and method manages medication delivery failures. An effective therapeutic range of a patient physiological property is determined based on a pharmacokinetic profile of a medication. During an administration of the medication to the patient, an expected trend in the measured physiological property is determined based on sensor data, the pharmacokinetic profile of the medication, a dose of the medication provided to the patient, and the at least one physical property of the patient, and an infusion device is caused to adjust the dose of the medication to cause the physiological property to follow the expected trend. Responsive to determining that a current trend in the physiological property has deviated from the expected trend, and will fall outside the effective therapeutic range within a predetermined time period, a delivery failure is determined and an alarm is provided.

### TECHNICAL FIELD

The present disclosure is generally related to managing and preventing medication delivery failures.

### BACKGROUND

Medication flow interruption may be caused by an intravenous (IV) line occlusion or a line disconnect. Some typical causes include: closed line clamps, kinked or pinched lines, luer lock leaks and other line leaks, valves in lines becoming disconnected or closed. These may not be apparent to the clinician. Currently only occluded lines are detected and produce an alarm by the infusion pump. Moreover, compliance in an IV set and partial occlusions as well as system variability can result in inaccurate systems that cause nuisance alarms, or alarm too late hindering the patient treatment. Leakage of medication is not currently detected by pumps.

### SUMMARY

According to various aspects, the subject technology provides a system and method for managing medication delivery failures. In this regard, a medical device resource management system is disclosed for use with infusion pumps and related devices.

According to various aspects, a method includes determining, for an administration of a medication to a patient by an infusion device, an effective therapeutic range of a physiological property of the patient based on a pharmacokinetic profile of the medication, a dose of the medication provided to the patient, and at least one physical property of the patient; determining, during the administration of a medication, an expected trend in the physiological property based on first sensor data from a sensor for a prior period of time, the pharmacokinetic profile of the medication, the dose of the medication provided to the patient, and the at least one physical property of the patient; causing the infusion device to adjust the dose of the medication to cause the physiological property to follow the expected trend; determining, after the dose is adjusted by the infusion device to follow the expected trend, that a current trend in the physiological property deviated from the expected trend and that the current trend will cause the physiological property to fall outside the effective therapeutic range within a predetermined time period; and responsive to determining that the current trend will cause the physiological property to fall outside the effective therapeutic range within the predetermined period of time: determining a delivery failure of the medication based on the current trend; and providing an alarm of the delivery failure. Other aspects include corresponding systems, apparatus (e.g., an infusion device), and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example of an institutional patient care system of a healthcare organization, according to aspects of the subject technology.

FIG. 2 is a conceptual diagram illustrating an example system architecture, including an example resource management unit, according to aspects of the subject technology.

FIG. 3A is a graph of an example biometric sensor measurements over time, according to various aspects of the subject technology.

FIG. 3B is a graph of the pharmacokinetic profile of a first example medication with respect to the medication's effective therapeutic range for an example patient, according to various aspects of the subject technology.

FIG. 3C is a graph of the pharmacokinetic profile of a second example medication with respect to the medication's effective therapeutic range for an example patient, according to various aspects of the subject technology.

FIG. 4A depicts a first example process for managing medication delivery failures, according to aspects of the subject technology.

FIG. 4B depicts a second example process for managing medication delivery failures, according to aspects of the subject technology.

FIG. 5 is a conceptual diagram illustrating an example electronic system for managing medication delivery failures, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Infusion pump systems provide a means to detect an occlusion. However, in cases where the infusion flow rate is low, the detection may take a very long time before alerting the clinician. In such cases, the alert may come at a time where it may take a significant amount of time to stabilize the patient's condition. Currently there are no known means to detect (without visual inspection) set leakage or other types of interruptions such as a kinked or partially occluded intravenous (IV) infusion line not delivering the required quantity of medication to the patient.

According to various implementations described herein, an external infusion control device operably connects to one or more infusion devices and one or more sensors, and is configured to control and/or facilitate operation of such devices to manage medication delivery failures. The control device, and corresponding systems and methods, control the infusion device(s) based on sensor data provided by the sensors and user input provided at the control device. The control device provides a user interface to a display and, on receiving a therapy selected by a user, confirms the sensors for the therapy are operably connected to the control device and one or more algorithms control the infusion device(s) based on the sensor data. Performance of the selected therapy is facilitated by the control device based on sensor data received from the one or more sensor devices, which are further used to determine an infusion failure. By monitoring both biometric sensors and the medication input to the patient, the subject technology alerts the clinician well before a critical condition occurs, thus preventing adverse effects to the patient.

According to various implementations, the control device may be preloaded with, or obtain (e.g., via a server), profiles for various fluids (including, e.g., medication(s)). An effective range (e.g., an effective therapeutic range) of a patient property measured and/or received by the control device (e.g., a patient physiological property or other measured property) is determined based on one or more first inputs, including a predetermined profile of a fluid (e.g., a pharmacokinetic profile of the medication) being delivered to a target (e.g., a patient) by an infusion pump operably connected to the control device. During delivery of the fluid to the target by the infusion pump, an expected trend in the measured property is determined based one or more second inputs, including sensor data, the predetermined profile, an amount (e.g., dose) of the fluid being delivered to the target, and the at least one physical property of the target, and the infusion pump is caused to adjust the amount of the fluid being delivered to the target to cause the measured property to follow the expected trend. Responsive to determining that a current trend in the measured property has deviated from the expected trend, the data may be reanalyzed (e.g., by the control device) and, if the current trend is determined to fall outside the effective range within a predetermined time period, a delivery failure may be determined and an alarm provided.

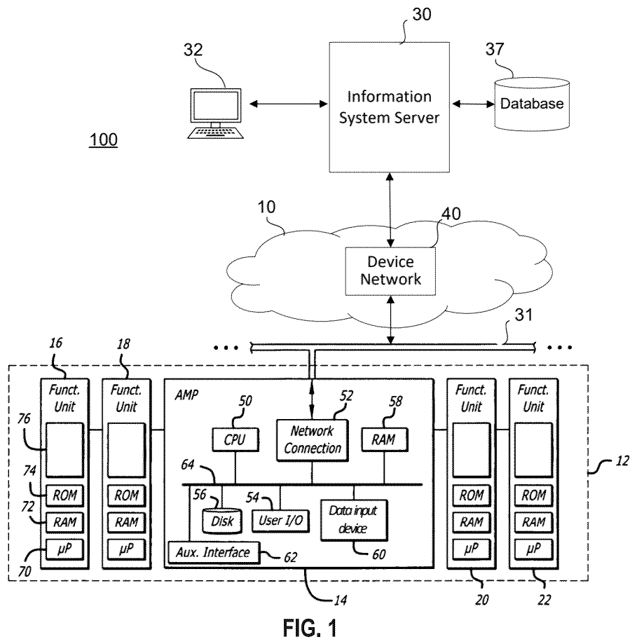

FIG. 1 depicts an example of an institutional patient care system 100 of a healthcare organization, according to aspects of the subject technology. In FIG. 1, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 may be a wired or wireless transmission channel, for example an 802.11 wireless local area network (LAN). In some implementations, network 10 also includes computer systems located in various departments throughout a hospital. For example, network 10 of FIG. 1 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 40 by which patient care devices 12 (and other devices) communicate in accordance with normal operations.

Additionally, institutional patient care system 100 may incorporate a separate information system server 30, the function of which will be described in more detail below. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistants, mobile devices such as laptops, tablet computers, augmented reality devices, or smartphones, configured with software for communications with information system server 30 via network 10.

Patient care device 12 comprises a system for providing patient care, such as that described in U.S. Pat. No. 5,713,856 to Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a interface device 14, also referred to as interface unit 14, connected to one or more functional modules 16, 18, 20, 22. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include means (specifically configured with one or more features described) for displaying and inputting information, such as a monitor, a printer, a keyboard, softkeys, a mouse, a track ball and/or a light pen. Data input device 60 may be a bar code reader capable of scanning and interpreting data printed in bar coded format. Additionally, or in the alternative, data input device 60 can be a specifically configured device for entering coded data into a computer, such as a device(s) for reading a magnetic strip, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1 to be disposed within interface unit 14, it is recognized that data input device 60 may be integral within pharmacy system 34 or located externally and communicating with pharmacy system 34 through an RS-232 serial interface or other appropriate communication means. Auxiliary interface 62 may be an RS-232 communications interface, however other means for communicating with a peripheral device in the described environments such as a printer, patient monitor, infusion pump or other medical device may be used without departing from the subject technology. Additionally, data input device 60 may be a separate functional module, such as modules 16, 18, 20 and 22, and configured to communicate with controller 14, or other systems on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem, or a cable modem. Direct or indirect network connection may be used, including, but not limited to a telephone modem, an MIB system, an RS232 interface, an auxiliary interface, an optical link, an infrared link, a radio frequency link, a microwave link, a personal area network connection, a local area network connection, a cellular link, or a WLANS connection or other wireless connection.

Functional modules 16, 18, 20, 22 are specially configured devices for providing care to a patient or for monitoring patient condition. As shown in FIG. 1, at least one of functional modules 16, 18, 20, 22 may be an infusion pump module such as an intravenous infusion pump for delivering medication or other fluid to a patient. For the purposes of this discussion, functional module 16 is an infusion pump module. Each of functional modules 18, 20, 22 may be a patient treatment or monitoring device including, but not limited to, an infusion pump, a syringe pump, a patient-controlled analgesia (PCA) pump, an epidural pump, an enteral pump, a blood pressure monitor, a pulse oximeter, an EKG monitor, an EEG monitor, a heart rate monitor, or an intracranial pressure monitor or the like. Functional module 18, 20 and/or 22 may be a printer, scanner, bar code reader or other peripheral input, output, or input/output device.

Each functional module 16, 18, 20, 22 communicates directly or indirectly with interface unit 14, with interface unit 14 providing overall monitoring and control of device 12. Functional modules 16, 18, 20, 22 may be connected physically and electronically in serial fashion to one or both ends of interface unit 14 as shown in FIG. 1, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1, any number of devices may be connected directly or indirectly to central controller 14. The number and type of functional modules described herein are intended to be illustrative, and in no way limit the scope of the subject technology. Module-specific components 76 include specifically configured components necessary for operation of a particular module, such as a pumping mechanism for infusion pump module 16.

While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics, or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction, or a combination of both automated and manual interaction. Automated interaction may be continuous or intermittent and may occur through direct network connection 54 (as shown in FIG. 1), or through RS232 links, MIB systems, RF links such as BLUETOOTH, IR links, PANS, LANS, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or other media for storing data. The communication means in various aspects is bidirectional with access to data from as many points of the distributed data sources as possible. Decision-making can occur at a variety of places within network 10. For example, and not by way of limitation, decisions can be made in HIS server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication

In some implementations, medication delivery modules 16, 18, 20, 22 include plug-in ports for expansion. Accordingly, a new medication delivery module may be attached to PCU 12 by coupling a connector through the plug-in ports, which may include electrical terminals so that the added medication delivery module 16, 18, 20, 22 may transmit and receive information to and from a control module 14. In some implementations, the added medication delivery module 16, 18, 20, 22 may also receive power from control module 14 through a plug-in port. Control module 14 may include a main display, a memory and a processor (see FIG. 10), and may be configured to display operational parameters and medication delivery status, and further information associated with each of medication delivery modules 16, 18, 20, 22. According to various implementations, module displays may also display physiological data (e.g., vital signs) associated with a patient.

A main display (e.g., I/O 54) may be configured to display one or more user interfaces for the display of operational parameters or other data associated with a module 16, 18, 20, 22, and/or physiological properties associated with the patient. The main display may include multiple user interfaces, with each individual user interface graphically displaying information for a respective one of medication modules, including information also displayed on a corresponding module displays. In some implementations, control module 14 includes a communications module (including, e.g., an antenna), configured to communicate wirelessly with a controller, or with a network.

With reference to FIG. 1, when a medication delivery module 16, 18, 20, 22 initiates an infusion of a medication to the patient, the control module 14 is configured to create and manage an infusion session within a memory of the control module (or related module). For the purpose of this disclosure, the infusion session includes state information of the PCU 12, its control module 14, and/or its associated modules, which is recorded and saved to memory during a particular period of time. The state information includes, but is not limited to, records of parameter values utilized by the PCU, its control module, and/or its associated modules during the period of time, and/or records physiological data collected during the period of time. During the infusion, physiological data associated with the patient is recorded within the session, operating parameter values, and modifications to the operating parameters of the PCU, its control module, and/or modules are also recorded in the session.

If not already logged into the PCU 12, the clinician may scan his or her badge proximate to a sensor (e.g., 54, 60) on the PCU 12, and the PCU may attempt to authenticate the clinician by sending the clinician's scanned identification to server 30. The clinician's badge may incorporate a radio frequency identification device (RFID), which is read by a scanner integrated with the PCU, or a portable scanner associated with the PCU. The clinician may scan his or her badge at the control module 14 to identify and authorize the clinician to initiate the administration of a medication. Once the clinician is associated with the PCU and/or module(s), the clinician's identification is associated with the session. The same is applicable with a patient. The clinician may scan the patient's wristband with a portable scanner, or using the sensor on the PCU 14 (or its control module) to associate the patient with the PCU and/or module(s) (and a session).

The control unit 14 of PCU 12 is configured to generate a graphical representation of the infusion session, and display (e.g., in a display) the graphical representation, including a graphical visualization of all parameters of the infusion during the session and modifications to the parameters, together with physiological data obtained during the session. The graphical representation may include pseudo identifiers for unknown data until such data is substituted with known identifiers. At that time, the graphical representation is displayed with the known patient identifiers

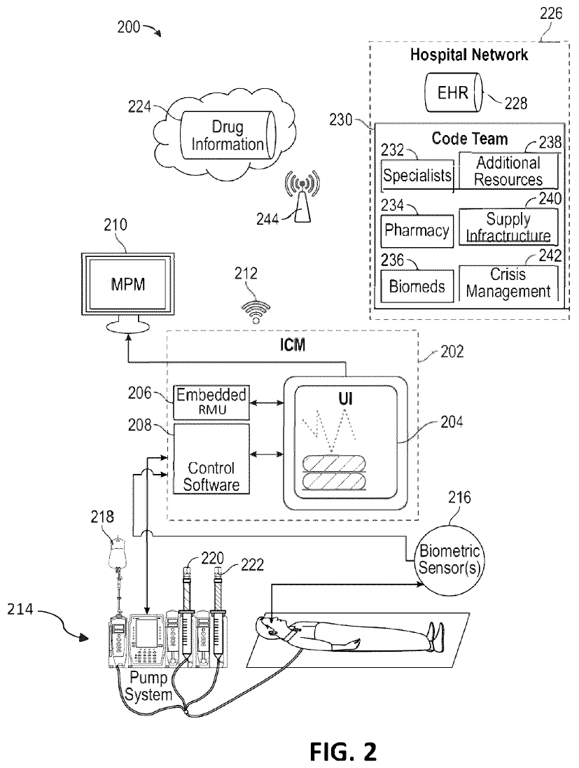

FIG. 2 is a conceptual diagram illustrating an example system architecture, including an example resource management unit, according to aspects of the subject technology. In the depicted example, a care provision system 200 includes an intelligent control module (ICM) 202. The ICM 202 provides direct control over connected medical devices to assist the clinician in providing a more centralized control and management of the devices. The ICM 202 provides a modular interface system by which various biometric sensors 216 used in a medical environment may be connected in a generic way to facilitate control over the connected medical devices. For example, the biometric sensors 216 may include one or more of a heart rate monitor, an oxygen sensor, and an intravenous (IV) flow rate monitor, all of which may be connected to the ICM 202 to facilitate, in addition to input by the clinician, centralized control of one or more infusion devices. As will be described further, the ICM 202 may be connected to a bispectral index (BIS) sensor 216 to assess the depth of anesthesia during an operation.

The ICM 202 may further connect to a server and/or cloud-based system for further data input, data coordination, and reporting. Examples of cloud-based systems include a cloud-based drug information database 224 (or formulary), and a hospital network 226 that includes electronic medical record (EMR) system or database 228.

The hospital network 226 may also include a code team 230 system and/or network that responds to code alarms. The code team 230 includes specialists 232 (human or AI), a pharmacy 234, biomedical technicians 236 (human or AI), crisis management resources 242, supply infrastructure 240, and additional resources 238 for responding to requests made to the code team 230.

In the depicted example, the ICM 202 includes a control unit 208, including control software, that provides processing for control algorithms, and connectivity circuitry and/or software to enable closed and semi-closed-loop control capabilities over one or medical devices at a point of use. In some implementations, the ICM 202 provides an external interface, for example a user interface 204, for interaction between an IV infusion pump 214 and one or more different physiological or biometric sensors 216, and for providing input parameters that may be used to control the titration of IV infusions of medications to a patient.

In some implementations, the closed-loop system provides control for the IV infusion of the medication by an infusion device 214 according to the feedback provided by the biometric sensors 216 used to monitor the therapy being performed. In situations where the IV medication is not being delivered as a result of abnormal conditions such as the IV line being occluded, leaking, or not being connected to the patient, the sensors 216 will indicate that the patient is not responding to the therapy. The subject technology provides feedback (e.g., a notification or alarm) to the clinician to alert the clinician of a possible fault condition that should be addressed, in some instances before a critical situation occurs.

The ICM 202 can incorporate control software (including, e.g., one or more algorithms in the unit 208) that can be tailored to specific or general medical treatments. The ICM 202 may receive infusion status information from infusion device 214. The infusion status information may include, for example, an identification of the medication, a flow rate of an administration of medication to a patient by the infusion device, a VTBI (volume to be infused), delivery duration, upstream or downstream pressure, and the like. While the infusion device 214 may have its own safety system, the ICM 202 may be preprogrammed to determine safety events such as events specific to a particular procedure. The ICM 202 may determine, based on sensor data from sensor(s) 216 and infusion status information, that a safety event is likely to occur within a predetermined period of time (e.g., programmed into the ICM or determined by AI based on training models for the procedure being undertaken).

A closed-loop control system as described herein generally refers to a system that does not rely on external manual inputs to deliver a therapy. Once configured, the closed-loop system can autonomously provide a therapy, receive feedback from one or more sensors 216 and, based on the feedback, automatically adjust the therapy as needed. For example, the ICM 202 may determine, during an administration of a medication, an expected trend in a physiological property during a predetermined time period based on sensor data for a prior period of time, a dose of the medication provided to the patient, and the one or more physical parameters of the patient. The ICM 202 may then cause the infusion device to adjust the dose of the medication to cause the physiological property to follow an expected trend within a predetermined time period.

Having the ICM as a separate unit from the medical device provides several advantages. For example, the advancement of sensors (e.g., biometric sensor(s) 216) may be much more rapid than the development of infusion pump systems (e.g., pump system 214), and thus the control system may accommodate these changes more quickly. It may be desirable to update control algorithms to address changes in treatment methods, available medications, and patient physiology. For this reason, it may be desirable to have the algorithm reside in a component different than the pump system, in order to accommodate more frequent changes. Moreover, machine learning and artificial intelligence may account for patient variations related to physiological properties such as age, genetics, health history, and other characteristic and environmental factors. Systems incorporating such capabilities may involve large databases and complex programs requiring powerful microprocessors and data storage capabilities to perform the timely and accurate computation needed. These systems are generally not capable of running on the systems currently available with the IV pumps alone, but may reside on a server 30 or other cloud-based system accessible from the ICM.

Additionally, the ICM 202 of the subject technology may be adaptable to a variety of pump systems and sensor inputs. The ICM 202 may further be configured to add wireless, Bluetooth and LAN connections to pump systems that do not currently have it available. For example, the FIG. 2 shows the ICM 202 having a wireless connection 212 for communication with a network system 244 at the hospital. Adding such communications to the pump system 214 may enable other capabilities such as remote monitoring and control of the infusion pumps, and facilitating access to EMR 228. Accordingly, by integrating the electrical and processing components separate from the pump, the subject technology facilitates integration of additional capabilities without needing to modify the pump's housing and electronics. Separating the physiological sensing and control systems from the infusion pump system may further provide for a more streamlined regulatory approval process.

According to various implementations, the ICM 202 facilitates the control software 208 to be separate from the embedded firmware 206 of the pumps and from the sensors, which can facilitate a scalable and rapidly configured system to provide closed-loop control of medical treatments. Moreover, the ICM 202 can be configured to operate with a multitude of sensors by way of electrical connectors and/or wireless communication. The ICM 202 may include one or more microprocessors and algorithms to provide signal conditioning and/or conversion of the sensor signals to the appropriate physiological properties for the connected medical device. The parameters may then be used in control algorithms to provide control to, for example, an infusion pump to deliver the necessary medications or fluids for a desired clinical outcome. As used herein, “connecting” devices or “operably connecting” devices may include establishing a physical (e.g., wired) or virtual (e.g., wireless) connection between the devices.

The user interface 204 of ICM 202 can include a display module or a touch-sensitive display module configured to provide a user interface for display of information pertaining to patient physiological status, as well as system control status. In some implementations, the user interface 204 includes circuitry within the ICM 202 housing that provides display information to an external display device. An example of such an external display device includes a multi-parameter monitor (MPM) 210. The MPM 210 may, for example, display various vital statistics (e.g., electrocardiogramaxygen saturation level (SpO2)) of a patient in a surgery room such that the vital statistics are visible to the clinicians (e.g., all the clinicians) in the room. The display on the ICM 202 may be mirrored via an associated MPM 210 to provide information to devices connected to the MPM 210 and/or clinicians involved in the treatment.

The ability of the ICM 202 to connect to another display (e.g., the MPM 210) provides modular scalability. For example, a more extensive user interface may be beneficial in some use cases. The more extensive user interface may include displays of data and graphs, for which a larger high-resolution display may be used. Some use cases may benefit from a display that have minimal information and a corresponding user interface to support such a configuration. A smaller, space saving and/or lower cost locally connected display may then be used.

According to various implementations, the ICM 202 includes a resource management unit (RMU) 206. The RMU 206 stores protocol information (e.g., information about processes and steps associated with therapies and patients), making the information readily accessible in the ICM 202. In some implementations, the RMU 206 provides information to the user interface 204, allowing the system to more efficiently (e.g., using fewer device resources such as memory, power, processing cycles, user interface area, and the like) present and navigate through pertinent information (e.g., using touch inputs on a touch screen). According to variously implementations, the RMU 206 guides the user to the order of steps and information needed at the appropriate time thereby avoiding expenditures of resources on information that is untimely or irrelevant to the currently detected condition.

In some implementations, the ICM 202 may access additional resources through the communication capabilities of the RMU 206. For example, the RMU 206 may access additional resources relating to medication information and dosage calculations from an internal or remote storage (e.g., from the drug information database 224, from a centralized). In some implementations, the RMU 206 can cause the user interface 204, and/or other operably connected user devices to display a dosage calculator for a patient when a particular medication is to be administered. In some implementations, the dosage calculator is a weight-based calculator that accounts for a weight of the patient (e.g., the weight of the patient is provided to the ICM 202 by manual entry by a clinician, or based on information received from the EHR database 228). In some implementations, calculations for weight-based medications can be readily accessed through the user interface 204 or the one or more user devices communicatively connected to the ICM 202. A patient's electronic health record may also be readily accessed directly through the ICM 202 to provide critical health and allergy information to the clinicians. The additional resources relating to medication information accessed by the ICM 202 via the RMU 206 may include information about a compatibility of medications that a patient is prescribed to receive. In some implementations, the RMU 206 can cause the user interface 204, and/or other user devices to display mixing ratio instructions. For example, the RMU 206 may cause the user interface 204 to specify a current or programmed flow rate of the first fluid from the syringe pump 220, a current or programmed flow rate of the second fluid from the syringe pump 222, and a current or programmed flow rate of the infusion fluid from the infusion bag 218 to facilitate an appropriate mixture for administration to the patient during the infusion.

In one example, the RMU 206 may access additional resources by allowing a clinician to electronically deliver or make pharmacy requests via the ICM 202 to the pharmacy 234. The RMU 206 can cause the user interface 204 to display controls that allow a clinician to summon one or more specialists for consultation (e.g., virtually using cameras communicatively connected to the ICM 202 and/or data from the biometric sensors 216; or request for an in-person consultation at the patient's location). In some implementations, the RMU 206 may connect the clinician to an AI. The RMU 206 can cause the user interface 204 to display controls that allow a clinician to send code alarms so that additional resources and personnel (e.g., human or AI) can respond to a medical issue. The RMU 206 can cause the user interface 204 to display controls for equipment requests or for other resources needed by the patient. Thus, in the cases where a hospital code would need to be issued, a clinician can simply use the RMU 206 to bring up checklists that would then present appropriate codes available for specific requests to be sent via the hospital network 244. In some implementations, the user interface may include a control element that, when activated, transmits a control message to a device in communication with the RMU 206 to request a resource.

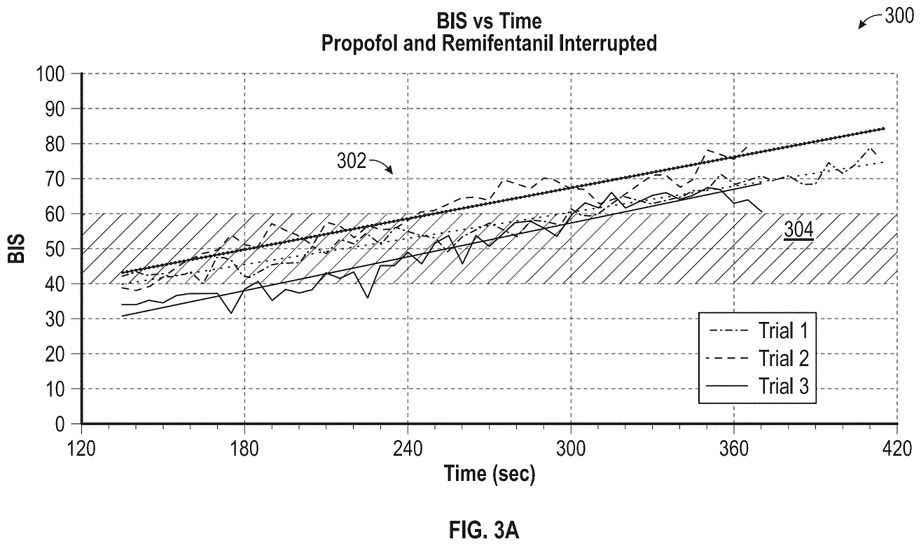

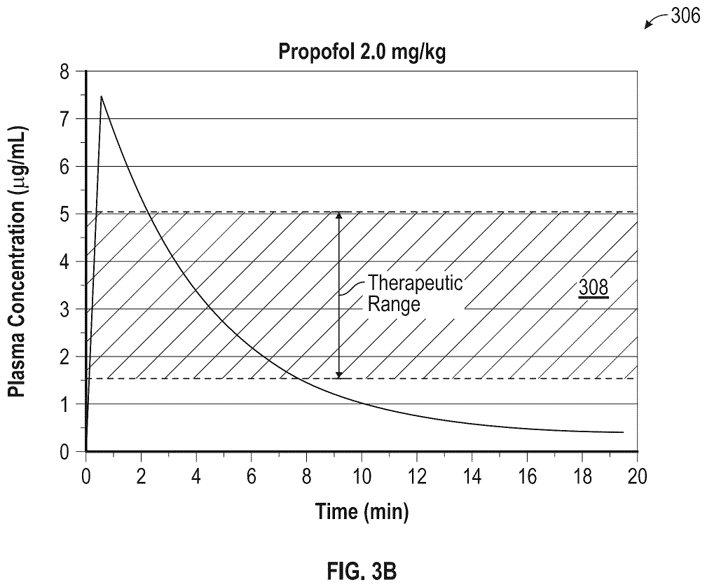

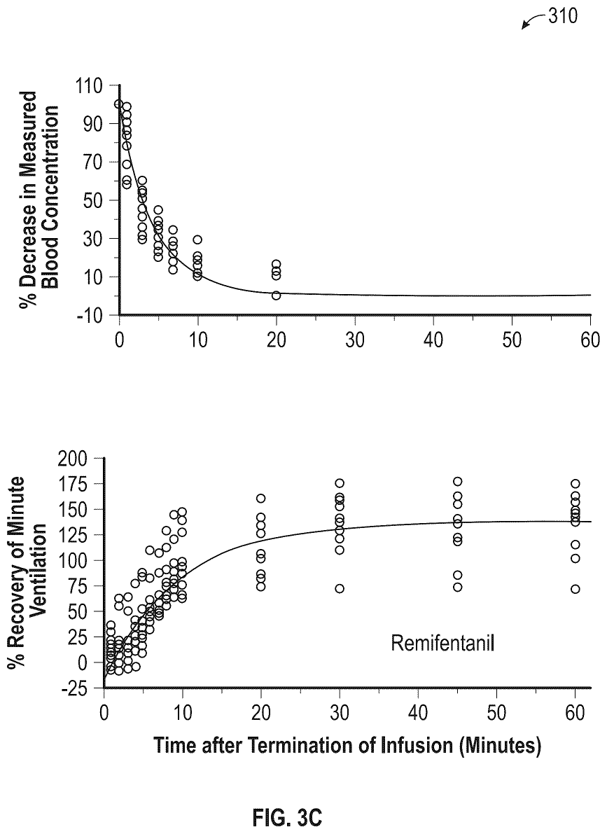

FIG. 3A is a graph 300 of example trends 302 in biometric sensor measurements over time, according to various aspects of the subject technology. In various implementations, the disclosed system monitors one or more biometric sensors and trends the sensor's direction in connection with monitoring an administration of medication that is tied into the biometric parameter through the closed loop control system. Upper and lower limits (e.g., defined by the shaded area 304) can be set to indicate whether the patient's response is following the expect results from the medication being administered. The system may perform a regression analysis on the sensor data to determine an expected trend over a period of time. By trending the biometric parameter and predicting when it will reach a critical limit an alarm can be provided to the clinician to advise them to inspect IV lines to ensure that medication delivery is not obstructed or not being delivered to the patient.

In this regard, the system of the subject technology may determine a trend in a physiological property (e.g., by way of sensor measurements) and, based on the trend, estimate a value of the physiological property (e.g., a future measurement) at a future time, and/or estimate a time when a safety event is likely to occur. For the purpose of this disclosure, a safety event may include, for example, a point in time in which a drug administered to the patient no longer has its intended pharmacokinetic effect. In the depicted example, the trend is based on bispectral index (BIS) sensor measurements. Where the administered drug(s) includes an anesthesia, the safety event may include when the patient would be expected to begin to awaken and/or exhibit pain.

The BIS sensor monitors the patient's depth of anesthesia. The BIS may incorporate other parameters such as the EMG (Electromyography) and BSR (Burst Suppression Ratio). The BIS sensor readings are sampled periodically (e.g., at approximately 10 second intervals) and tracked over the duration of the procedure. The signal can then be used to trend BIS measurements using regression analysis and predict at what point the BIS will reach a target level of interest. Therefore, by monitoring both the increase in BIS and the delivery of a medication or combination of medications (e.g., propofol and remifentanil) over time it can be determined that the medication(s) may not be reaching the patient.

The slope of the BIS value over time is calculated.

$$\frac{d BIS}{dt}$$

If the value is increasing, it can be determined at what point in time the BIS value will increase to reach a certain level of concern for patient awareness. In closed loop implementations, the closed loop control will try to adjust for this increasing BIS value by increasing the infusion of the propofol and remifentanil. However, in a situation where the line is obstructed or leaking, the patient will not be receiving the medication and the BIS value will not be decreasing. Therefore, by monitoring both the increase in BIS and medication over time it can be deduced that the medication is not reaching the patient.

The BIS signal may contain significant noise and artifacts. In order to reduce the effect of noise or variability in the BIS signal, an IIR filter is used to “clean” the signal which results in a reduced sensitivity to false alarms.

The depicted example plots simulated tests where the BIS value was increasing as a result of flow interruption, despite the fact that the control system was trying to increase the infusion of propofol and remifentanil up to the maximum Ce (concentration effect) levels of 8.0 and 4.0 respectively. According to the depicted example Trial 1 data, when the BIS reaches a value of 55 it would take approximately another minute to reach a value of 60. Patient awareness may start to begin at this level.

From tests performed with a simulator, the effect on BIS values from the medications propofol and remifentanil diminish after about 2 minutes (illustrated by dips along the regression lines). And after resuming the administration of the drugs, BIS measurements return to nominal (<60 levels) within 1 minute. It is understood that the amount of time a patient stays within nominal levels is dependent on the patient characteristics.

FIG. 3B is a graph 306 of the pharmacokinetic profile of a first example medication with respect to the medication's effective therapeutic range 308 for an example patient, according to various aspects of the subject technology. A pharmacokinetic profile for propofol is depicted. According to various implementations, the pharmacokinetic profile indicates when a blood (or plasma) concentration of the given medication will reach a target level after a dose of the medication is provided and/or how long the blood (or plasma) concentration will remain at the target level. According to various implementations, the target level may be a predetermined range.

In the depicted example, the pharmacokinetic profile indicates that the concentration of propofol required to maintain a patient within the proper therapeutic range 308 for anesthesia is between 1.5 μg/mL and 5 μg/mL, and 2.0 mg/kg of propofol will maintain a patient within the therapeutic range for approximately 8 minutes. When the patient's blood concentration measures within this range the patient is expected to be anesthetized. It is understood herein that the therapeutic range may vary from patient to patient and, in this regard, the pharmacokinetic profile of a given medication may be based not only on the medication, but also may be based on patient physical and physiological properties. In the depicted example, the target blood concentration is achieved (and exceeded briefly) in under a minute.

FIG. 3C is a graph of the pharmacokinetic profile 310 of a second example medication with respect to the medication's effective therapeutic range for an example patient, according to various aspects of the subject technology. In FIG. 3C The top graph represents the best fit pharmacokinetic effect of Remifentanil for one or more example patients. After a dose is administered, Remifentanil may have a context-sensitive half-life time of 3 minutes (e.g. the point at which the drug concentration in the blood is 50%, after discontinuation of infusion of the drug). Accordingly, the top graph depicts the percentage decrease in measured blood concentration of Remifentanil for an example patient. The corresponding lower graph depicts an example recovery of minute ventilation (e.g., the measure of the patient's respiratory rate) for one or more example patients. The respiratory rate and/or minute ventilation can be used as an indicator of pain experienced by the patient. Minute ventilation can be monitored by CO2 or EtCo2 sensors and is another input to detect the corresponding interruption in analgesic (e.g. Remifentanil).

In the depicted examples, the percentage of decrease in measured blood concentration of the drug and percentage of recovery of minute ventilation (e.g., a measure of patient respiratory activity) are plotted against a time after termination of infusion for remifentanil. The depicted example illustrates that a patient should reach 100% recovery within 20 minutes after termination of a remifentanil infusion. The subject innovation may assess against a specific point on the recovery curve. For example, the remifentanil pump continues to administer the drug, but the patient exhibits ventilation recovery associated with 10 minutes after termination. In this case, the patient may not be receiving the remifentanil due to occlusion or leakage. In some implementations, multiple measurements for a patient parameter may be compared to an expected response. In such implementations, a single measurement failing to correspond with the expected threshold may not trigger a response, but multiple failures over a period of time may trigger a system response. (Here “failure” is used to reference a condition needing a corrective action). Typically, an occlusion or leak is a persistent condition that manifests until corrected. Such trend data may be useful to prevent the system from over responding to outlier or random, one time, patient responses that may not be related to occlusion or leak.

As user herein, the terms “correspond” or “corresponding” when referring to system information encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

The thresholds, such as the first threshold and/or the second threshold, described may be static, dynamic, or adaptive. Static thresholds are predetermined thresholds that remain constant. Dynamic thresholds are changed throughout operation of the system based on changes to the system or items stored therein. For example, as the need for the medication increases, it may be desirable to adjust the first threshold or the second threshold. A dynamic threshold may be based on a number of factors, including a need for the medication, a size of the medical facility, a location of the medical facility, or the like. A dynamic threshold may be based on time or date. For example, there may be times when there are fewer medication orders requested within an environment. Adaptive thresholds may be changed in response to changes in characteristics of the medical devices and/or items stored therein and may vary depending parameters detectable or received by the system. Whether static, dynamic, or adaptive, the threshold may be specified as a value or a range of values.

With reference to the pharmacokinetic properties shown in FIGS. 3B and 3C, tests performed with a simulator indicate that the effect on BIS from propofol and remifentanil diminish after about 2 minutes. And after resuming the administration of the drugs, BIS returned to nominal (<60 levels) within 1 minute. However, these are dependent on the patient characteristics.

The decision analysis may then follow the criteria below.
Where
   BISₜ₌₁=predicted BIS value rise in 1 minute, based on a regression analysis of the previous 2 minutes of BIS readings.
   P_{Ce}=Propofol Concentration Effect.
   R_{Ce}=Remifentanil Concentration Effect.
If
   BIS≥60 and P_{Ce}=maximum level setting (determined by anesthetist) and/or
   R_{Ce}=maximum level setting (determined by anesthetist)
   BISₜ₌₁=70
Then, issue an alert to check the IV line for occlusion or disconnection.

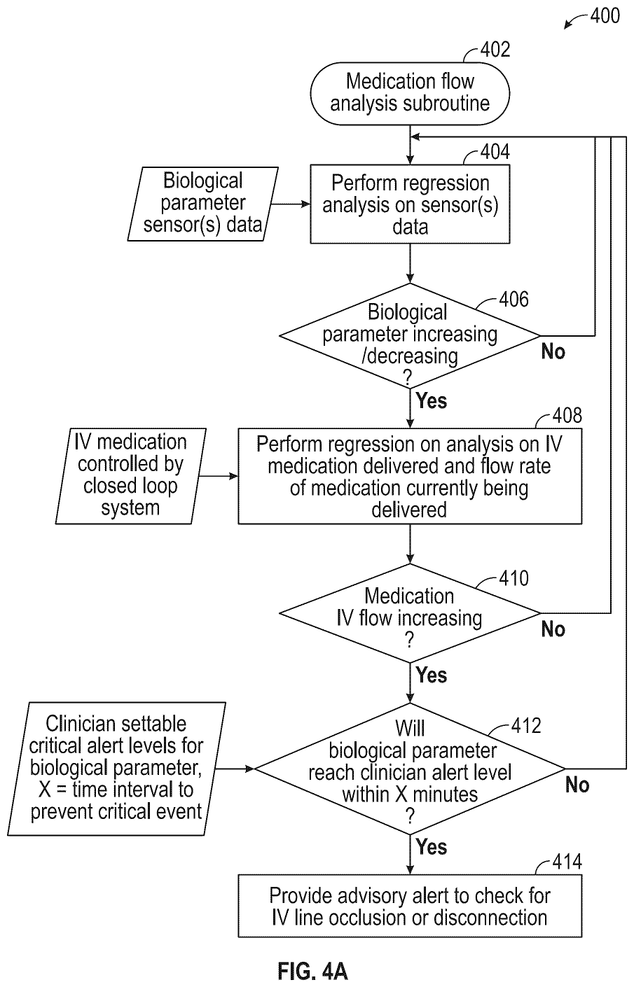

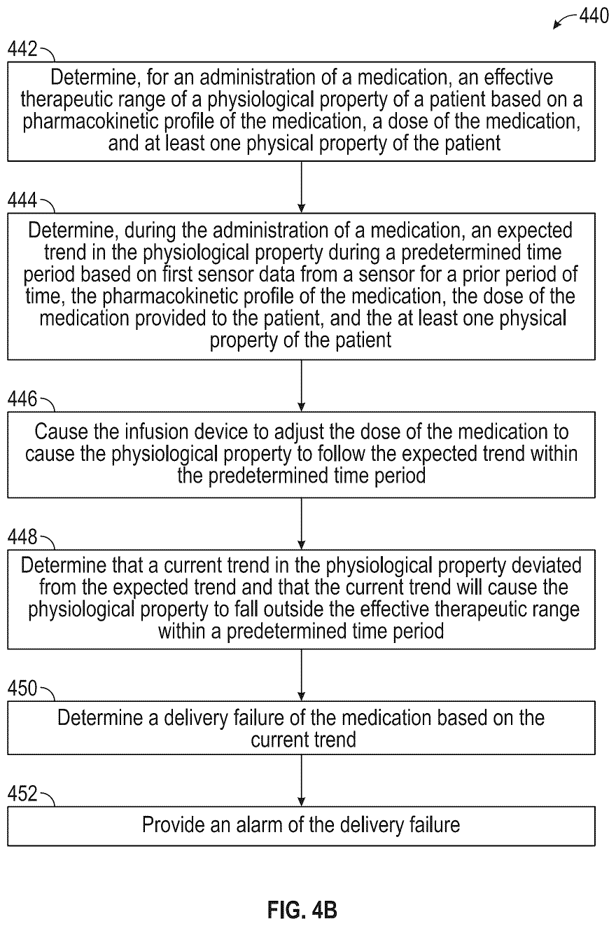

FIG. 4A depicts a first example process 400 for managing medication delivery failures, according to aspects of the subject technology. For explanatory purposes, the various blocks of example process 440 are described herein with reference to FIGS. 1 through 4A, and the components and/or processes described herein. The one or more of the blocks of process 440 may be implemented by a control device such as, for example, ICM 202 or a component thereof, interface 14, or one or more associated computing devices.

In the depicted example, a medication flow analysis subroutine is initiated (402) and a regression analysis is performed (404) on sensor data. According to various aspects, the sensor data includes a bispectral sensor measurements over a period of time. Based on the regression analysis, the system (e.g., the algorithm) determines whether a given physiological property (or parameters) is stable or unchanging (e.g., within a predetermined deviation of prior parameters for a period of time), or whether the parameters are increasing or decreasing (e.g., by way of being outside of the expected deviation) (406). According to various aspects described herein, physiological properties may be determined based on sensor measurements, and physiological properties and physiological measurements may be used interchangeably herein.

If the physiological properties are increasing or decreasing, the system performs regression analysis on one or more parameters of an IV medication being administered to the patient. In the depicted example, a regression analysis is performed on a flow rate of a currently administered medication (408). If the flow of the medication is not increasing then the process returns to the beginning (410) and performs further regression analysis on the sensor data. If the flow of the medication is increasing then the system determines whether the physiological property will reach an alert level within a predetermined period of time (412).

According to various implementations, the alert level may be representative of the physiological property falling outside of an effective therapeutic range for the medication. In some implementations, the therapeutic range is determined of the physiological property is determined based on the medication, one or more physical properties of the patient (e.g., weight, BMI, etc.), and a sensor type for measuring the physiological property (e.g., a BIS sensor). The system may determine, during administration of the medication, an expected trend in the physiological property based on a pharmacokinetic profile of the medication (see, e.g., FIG. 3B), sensor data from a sensor of the sensor type, a dose of the medication provided to the patient, and the one or more physical properties of the patient.

Prior to determining whether the physiological property will reach an alert level, the system may monitor the physiological property and attempt to adjust the dose of the medication to cause the physiological property to follow the expected trend. After the dose is adjusted (e.g., by the infusion device), the system may determine that the current trend will fall outside the effective therapeutic range within a predetermined period of time (e.g., within 1, 2, or 5 minutes).

If the system determines that the current trend will cause the physiological property to fall outside the effective therapeutic range then the system may provide an alarm (414). The alarm may indicate that a safety event has occurred. For example, a patient may be awakening from anesthesia, even though the infusion device continues to deliver the appropriate medication under the control of an anesthesiologist. An undetected occlusion or extravasation may have occurred that is preventing the anesthesia from reaching the patient. The alarm alerts the anesthesiologist (or other clinicians) of the safety event so that the problem can be immediately corrected before the patient is harmed.

FIG. 4B depicts a second example process 440 for managing medication delivery failures, according to aspects of the subject technology. For explanatory purposes, the various blocks of example process 440 are described herein with reference to FIGS. 1 through 4A, and the components and/or processes described herein. The one or more of the blocks of process 440 may be implemented by a control device such as, for example, ICM 202 or a component thereof, interface 14, or one or more associated computing devices. As previously described, the example process 440 may operate under control of AI, which may analyze sensor data and make any of decisions within example process 440 on behalf of the clinician. If a hard decision cannot be made (e.g., certain thresholds not being met) then the ICM 202 (including, e.g., a processor such as the RMU 206) may suggest a soft decision to the clinician who may then confirm, reject, or change the selection to treat the patient.

According to the depicted example process 700, the system determines, for an administration of a medication to a patient by an infusion device, an effective therapeutic range of a physiological property of the patient based on a pharmacokinetic profile of the medication, a dose of the medication provided to the patient, and at least one physical property of the patient (442).

The system determines, during the administration of a medication, an expected trend in the physiological property based on first sensor data from a sensor for a prior period of time, the pharmacokinetic profile of the medication, the dose of the medication provided to the patient, and the at least one physical property of the patient (444). According to various implementations, and as exemplified in FIGS. 3B and 3C, the pharmacokinetic profile indicates that a blood concentration of the medication will reach a target level within a predetermined time after the dose of the medication is provided to the patient. In this regard, the predetermined time period associated with the dose may be used to calculate a predetermined time period for which the expected trend is determined.

A regression analysis may be performed on historical physiological data over the course of a certain duration (e.g., the last measurements for the physiological property) to form the expected trend, which may then be analyzed into a future duration of time (e.g., the same duration) to determine whether the trend will fall outside of an effective sensor range. With reference to FIG. 3A, the sensor range 304 may be determined for the type of sensor used (e.g., BIS sensor) to indicate a range of values a given blood concentration within the therapeutic range of the medication (e.g., 308 as depicted in FIG. 3B). According to various implementations, the therapeutic range of a medication may determine a duration (as shown in FIG. 3B) that the medication is expected to be effective and, in some implementations, this duration may the duration over which the historical physiological data is analyzed.

In some implementations, the medication comprises an anesthesia drug, the sensor is a bispectral index (BIS) sensor and the effective therapeutic range is a range of the physiological property in which the patient is in a hypnotic state (e.g., under anesthesia). In some implementations, the medication includes a vasopressor, and the sensor is a biometric sensor configured to measure a cardiac output, and the effective therapeutic range is a range of the cardiac output in which the cardiac output is medically stable.

In some implementations, the predetermined time period may be set based on a current flow rate of the medication. As previously described, the flow rate may be included in the infusion status information obtained by the ICM 202 from the connected infusion device 214.

The system then causes the infusion device 214 to adjust the dose of the medication to cause the physiological property to follow the expected trend within the predetermined time period (446).

The system determines, after the dose is adjusted by the infusion device to follow the expected trend, that a current trend in the physiological property deviated from the expected trend and that the current trend will cause the physiological property to fall outside the effective therapeutic range within a predetermined time period (448). In this regard, the system may determine, based on second sensor data from the sensor, that the blood concentration will not reach the target level within a predetermined time period. In some implementations, the predetermined period of time for determining whether the current trend will fall outside the effective therapeutic range is received as input from a user input device. For example, the input may be from a touch enabled user input screen 204 of the ICM 202 or a connected input device, an input screen or device integrated with the infusion device 214, or may be from a user mobile device connected to the ICM 202 or infusion device 214.

Responsive to determining that the current trend will cause the physiological property to fall outside the effective therapeutic range, the system determines a delivery failure of the medication based on the current trend (450), and activates an output device to provide a human perceivable indication of the delivery failure, for example by providing an alarm of the delivery failure (452). In this regard, the system may be programmed to identify the delivery failure as an indication of an occlusion or a fluid line leak. Further analysis of the data may be performed to determine the nature of the failure. For example, on determining a failure has occurred, the system may activate a pressure sensor and determine from an increase in pressure that the failure results from an occlusion. A decrease in pressure may indicate the failure results from a fluid line leak.

Additionally or in the alternative to providing an alarm, on determining that the current trend will cause the physiological property to fall outside the effective therapeutic range, the closed-loop control algorithm of the ICM 202 may be configured to automatically adjust one or more connected infusion devices and/or pumps based on information such as the sensor measurements provided by one or more sensors 216 associated with a patient, sensors (and/or flow sensors) associated with the pump(s), and infusion information provided by the infusion pump(s). For example, the algorithm may be configured to maintain the patient at a particular therapeutic state (e.g., a hypnotic or sleep state) by controlling an amount of medication delivered by one or both of the pump(s) and to dynamically adjust the amount based on monitoring the patient with the sensors 216 for indications of how the patient is responding to the medication and/or how well the patient is stable within the therapeutic state. The amount of a medication may be controlled by the ICM 202 sending a control signal to the pump providing the medication to cause an adjustment to the pump's motor speed (e.g., to speed up or slow the motor and therefore the medication delivery). In some implementations, the motor speed may be slowed, reversed, and/or stopped to adjust or terminate delivery of the medication.

In some implementations, the system may identify (e.g., during the predetermined time period) an increase in a flow rate of the medication to correct the current trend and determine that the current trend was not corrected based on the increase in the flow rate. In this manner, the delivery failure may be determined based on the system not determining that the current trend was not corrected by the increase in the flow rate.

In some implementations, the system may receive (e.g., over the prior period of time) the sensor data from sensor 216 and infusion status information from the infusion device 214. The infusion status information may include a flow rate of a fluid being administered to a patient. The system may determine, based at least in part on the first sensor data and the infusion status information, that a safety event is likely to occur within the received predetermined time period. In such implementations, the alarm may indicate the safety event. In this regard, the increase in flow rate may be determined by the system performing a regression analysis on the infusion status information.

In some implementations, the medication administration may include more than one medication. For example, the medication administration may include a first drug and a second drug. In such implementations, the increase in the flow rate may pertain to the first drug and, responsive to determining that the current trend was not corrected, the system may adjust the flow rate of the second drug. The system may then obtain further sensor data from the sensor and determine whether the current trend was corrected based on the further sensor data. In this manner, the system attempts to compensate for a failure of the first drug by adjusting the flow of the second drug. For example, the system may be delivering propofol and remifentanil to a patient during a procedure and determine that the propofol drug has failed or otherwise became occluded. The system may then attempt to increase remifentanil to compensate for the lack of propofol and attempt to place the patient back into a deeper anesthesia (e.g., after determining that the patient was awakening based on data from sensor 216).

Many of the above-described example steps of processes 400 and 440, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

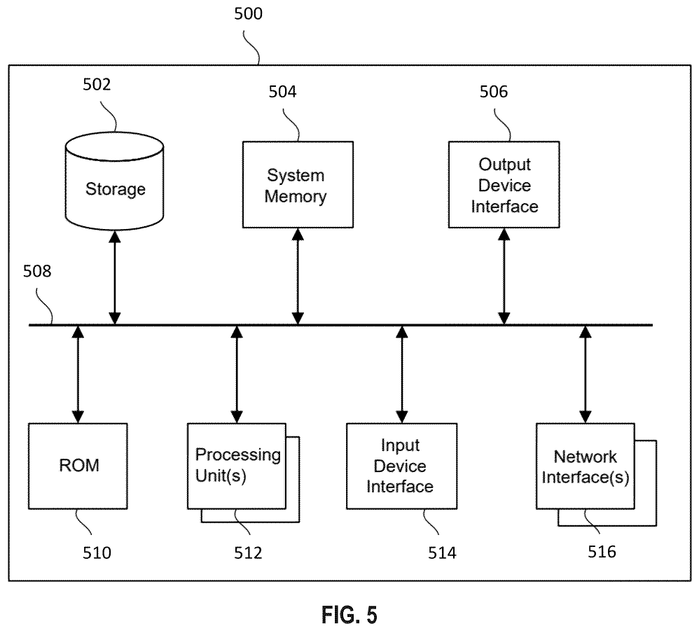

FIG. 5 is a conceptual diagram illustrating an example electronic system 500 for intelligent operation of infusion accessory devices, according to aspects of the subject technology. Electronic system 500 may be a specifically configured computing device for execution of software associated with one or more portions or steps of process 500, or components and processes provided by FIGS. 1 through 4, including but not limited to information system server 30, database 37, computing hardware within patient care device 12, or a remote device 32 (e.g., a mobile device). Electronic system 500 may be representative, in combination with the disclosure regarding FIGS. 1-5. In this regard, electronic system 500 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 500 may include various types of computer readable media and interfaces for various other types of computer readable media. In the depicted example, electronic system 500 includes a bus 508, processing unit(s) 512, a system memory 504, a read-only memory (ROM) 510, a permanent storage device 502, an input device interface 514, an output device interface 506, and one or more network interfaces 516. In some implementations, electronic system 500 may include or be integrated with other computing devices or circuitry for operation of the various components and processes previously described.

Bus 508 collectively represents all system, peripheral, and chipset buses that communicatively connect the numerous internal devices of electronic system 500. For instance, bus 508 communicatively connects processing unit(s) 512 with ROM 510, system memory 504, and permanent storage device 502.

From these various memory units, processing unit(s) 512 retrieves instructions to execute and data to process, in order to execute the processes of the subject disclosure. The processing unit(s) can be a single processor or a multi-core processor in different implementations.

ROM 510 stores static data and instructions that are needed by processing unit(s) 512 and other modules of the electronic system. Permanent storage device 502, on the other hand, is a read-and-write memory device. This device is a non-volatile memory unit that stores instructions and data even when electronic system 500 is off. Some implementations of the subject disclosure use a mass-storage device (such as a magnetic or optical disk and its corresponding disk drive) as permanent storage device 502.

Other implementations use a removable storage device (such as a floppy disk, flash drive, and its corresponding disk drive) as permanent storage device 502. Like permanent storage device 502, system memory 504 is a read-and-write memory device. However, unlike storage device 502, system memory 504 is a volatile read-and-write memory, such as a random access memory. System memory 504 stores some of the instructions and data that the processor needs at runtime. In some implementations, the processes of the subject disclosure are stored in system memory 504, permanent storage device 502, and/or ROM 510. From these various memory units, processing unit(s) 512 retrieves instructions to execute and data to process in order to execute the processes of some implementations.

Bus 508 also connects to input and output device interfaces 514 and 506. Input device interface 514 enables the user to communicate information and select commands to the electronic system. Input devices used with input device interface 514 include, e.g., alphanumeric keyboards and pointing devices (also called “cursor control devices”). Output device interfaces 506 enables, e.g., the display of images generated by the electronic system 500. Output devices used with output device interface 506 include, e.g., printers and display devices, such as cathode ray tubes (CRT) or liquid crystal displays (LCD). Some implementations include devices such as a touchscreen that functions as both input and output devices.

Also, as shown in FIG. 5, bus 508 also couples electronic system 500 to a network (not shown) through network interfaces 516. Network interfaces 516 may include, e.g., a wireless access point (e.g., Bluetooth or WiFi) or radio circuitry for connecting to a wireless access point. Network interfaces 516 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 500 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to specifically configured electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.
Clause 1. A method, comprising: determining, for an administration of a medication to a patient by an infusion device, an effective therapeutic range of a physiological property of the patient based on a pharmacokinetic profile of the medication, a dose of the medication provided to the patient, and at least one physical property of the patient; determining, during the administration of a medication, an expected trend in the physiological property based on first sensor data from a sensor for a prior period of time, the pharmacokinetic profile of the medication, the dose of the medication provided to the patient, and the at least one physical property of the patient; causing the infusion device to adjust the dose of the medication to cause the physiological property to follow the expected trend; determining, after the dose is adjusted by the infusion device to follow the expected trend, that a current trend in the physiological property deviated from the expected trend and that the current trend will cause the physiological property to fall outside the effective therapeutic range within a predetermined time period; and responsive to determining that the current trend will cause the physiological property to fall outside the effective therapeutic range within the predetermined period of time: determining a delivery failure of the medication based on the current trend; and providing an alarm of the delivery failure.
Clause 2. The method of Clause 1, wherein the pharmacokinetic profile indicates that a blood concentration of the medication will reach a target level within the predetermined time after the dose of the medication is provided to the patient, and wherein determining that the current trend will cause the physiological property to fall outside the effective therapeutic range comprises: determining, based on second sensor data from the sensor, that the blood concentration will not reach the target level within the predetermined time period.
Clause 3. The method of Clause 1 or Clause 2, further comprising: identifying a current flow rate of the medication; and setting the predetermined time period based on the current flow rate of the medication.
Clause 4. The method of any one of Clauses 1-3, further comprising: identifying, during the predetermined time period, an increase in a flow rate of the medication to correct the current trend; and determining that the current trend was not corrected based on the increase in the flow rate, wherein determining the delivery failure is based on determining that the current trend was not corrected by the increase in the flow rate.
Clause 5. The method of Clause 4, further comprising: receiving the predetermined period of time from a user input device; receiving, over the prior period of time, the first sensor data from the sensor and infusion status information from the infusion device, wherein the increase in the flow rate is identified based on the infusion status information; and determining, based at least in part on the first sensor data and the infusion status information, that a safety event is likely to occur within the received predetermined time period, wherein the alarm indicates the safety event.
Clause 6. The method of Clause 4, wherein the medication includes a first drug and a second drug, and wherein the increase in the flow rate pertains to the first drug, the method further comprising, responsive to determining that the current trend was not corrected: adjusting the flow rate of the second drug; obtaining second sensor data from the sensor; and determining whether the current trend was corrected based on the second sensor data.
Clause 7. The method of Clause 4, further comprising: receiving, over the prior period of time, the first sensor data from the sensor and infusion status information from the infusion device; and performing regression analysis on the infusion status information to determine the increase in the flow rate.
Clause 8. The method of any one of Clauses 1-7, further comprises: performing regression analysis on the first sensor data to determine the expected trend.
Clause 9. The method of any one of Clauses 1-8, wherein the delivery failure comprises an occlusion, fluid line leak, or disconnected intravenous infusion line.
Clause 10. The method of any one of Clauses 1-9, wherein the medication comprises an anesthesia drug, the sensor is a bispectral sensor and the effective therapeutic range is a range of the physiological property in which the patient is in a hypnotic state.
Clause 11. The method of any one of Clauses 1-9, wherein the medication comprises a vasopressor, the sensor is a biometric sensor configured to measure a cardiac output, and the effective therapeutic range is a range of the cardiac output in which the cardiac output is medically stable.
Clause 12. The method of any one of Clauses 1-11, wherein the infusion device comprises a pump motor, and causing the infusion device to adjust the dose of the medication comprises adjusting a motor speed of the pump motor.
Clause 13. The method of Clause 12, responsive to determining that the current trend will cause the physiological property to fall outside the effective therapeutic range within the predetermined period of time, further adjusting the motor speed of the pump motor to adjust a delivery of the medication to the patient.
Clause 14. The method of Clause 13, wherein adjusting the delivery of the medication to the patient comprises terminating delivery of the medication.
Clause 15. An infusion device hub, comprising: at least two connection ports configured to communicatively connect to the infusion device and the sensor; and wherein the device hub is configured to perform a method according to any one of Clauses 1 through 14.
Clause 16. A system, comprising: one or more processors; a memory device comprising non-transitory computer-readable instructions that, when executed by the one or more processors, cause the one or more processors to perform a method according to any one of Clauses 1 through 14.
Clause 17. A memory device comprising non-transitory computer-readable instructions that, when executed by one or more processors, cause the one or more processors to perform a method according to any one of Clauses 1 through 14.
Clause 18. An infusion control device, comprising: one or more processors; a non-transitory machine readable memory storing instructions that, when executed by the one or more processors, cause the infusion control device to: determine, for delivery of a fluid by an infusion pump operably connected to the infusion control device, an effective range of a measured property associated with a target of the fluid based on a predetermined profile of the fluid, an amount of the fluid, and at least one physical property of the target; determine, while the fluid is delivered by the infusion pump, an expected trend in the measured property based on first sensor data from a sensor for a prior period of time, the predetermined profile of the fluid, the amount of the fluid being delivered by the infusion pump, and the at least one physical property of the target; cause the infusion pump to adjust the amount of the fluid being delivered to cause the measured property to follow the expected trend; determine, after the amount is adjusted, that a current trend in the measured property deviated from the expected trend and that the current trend will cause the measured property to fall outside the effective range within a predetermined time period; and responsive to determining that the current trend will cause the measured property to fall outside the effective range within the predetermined period of time: determine a delivery failure of the medication based on the current trend; and provide an alarm of the delivery failure.

### FURTHER CONSIDERATIONS

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms web page and server. The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. A method, comprising:
determining, for an administration of a medication to a patient by an infusion device, an effective therapeutic range of a physiological property of the patient based on a pharmacokinetic profile of the medication, a dose of the medication provided to the patient, and at least one physical property of the patient;
determining, during the administration of a medication, an expected trend in the physiological property based on first sensor data from a sensor for a prior period of time, the pharmacokinetic profile of the medication, the dose of the medication provided to the patient, and the at least one physical property of the patient;
causing the infusion device to adjust the dose of the medication to cause the physiological property to follow the expected trend;
determining, after the dose is adjusted by the infusion device to follow the expected trend, that a current trend in the physiological property deviated from the expected trend and that the current trend will cause the physiological property to fall outside the effective therapeutic range within a predetermined time period; and
responsive to determining that the current trend will cause the physiological property to fall outside the effective therapeutic range within the predetermined period of time:
determining a delivery failure of the medication based on the current trend; and
providing an alarm of the delivery failure.

2. The method of claim 1, wherein the pharmacokinetic profile indicates that a blood concentration of the medication will reach a target level within the predetermined time after the dose of the medication is provided to the patient, and wherein determining that the current trend will cause the physiological property to fall outside the effective therapeutic range comprises:
determining, based on second sensor data from the sensor, that the blood concentration will not reach the target level within the predetermined time period.

3. The method of claim 1, further comprising:
identifying a current flow rate of the medication; and
setting the predetermined time period based on the current flow rate of the medication.

4. The method of claim 1, further comprising:
identifying, during the predetermined time period, an increase in a flow rate of the medication to correct the current trend; and
determining that the current trend was not corrected based on the increase in the flow rate,
wherein determining the delivery failure is based on determining that the current trend was not corrected by the increase in the flow rate.

5. The method of claim 4, further comprising:
receiving the predetermined period of time from a user input device;
receiving, over the prior period of time, the first sensor data from the sensor and infusion status information from the infusion device, wherein the increase in the flow rate is identified based on the infusion status information; and
determining, based at least in part on the first sensor data and the infusion status information, that a safety event is likely to occur within the received predetermined time period, wherein the alarm indicates the safety event.

6. The method of claim 4, wherein the medication includes a first drug and a second drug, and wherein the increase in the flow rate pertains to the first drug, the method further comprising, responsive to determining that the current trend was not corrected:
adjusting the flow rate of the second drug;
obtaining second sensor data from the sensor; and
determining whether the current trend was corrected based on the second sensor data.

7. The method of claim 4, further comprising:
receiving, over the prior period of time, the first sensor data from the sensor and infusion status information from the infusion device; and
performing regression analysis on the infusion status information to determine the increase in the flow rate.

8. The method of claim 1, further comprises:
performing regression analysis on the first sensor data to determine the expected trend.

9. The method of claim 1, wherein the delivery failure comprises an occlusion, fluid line leak, or disconnected intravenous infusion line.

10. The method of claim 1, wherein the medication comprises an anesthesia drug, the sensor is a bispectral sensor and the effective therapeutic range is a range of the physiological property in which the patient is in a hypnotic state.

11. The method of claim 1, wherein the medication comprises a vasopressor, the sensor is a biometric sensor configured to measure a cardiac output, and the effective therapeutic range is a range of the cardiac output in which the cardiac output is medically stable.

12. The method of claim 1, wherein the infusion device comprises a pump motor, and causing the infusion device to adjust the dose of the medication comprises adjusting a motor speed of the pump motor.

13. The method of claim 12, responsive to determining that the current trend will cause the physiological property to fall outside the effective therapeutic range within the predetermined period of time, further adjusting the motor speed of the pump motor to adjust a delivery of the medication to the patient.

14. The method of claim 13, wherein adjusting the delivery of the medication to the patient comprises terminating delivery of the medication.

15. An infusion device hub, comprising:
at least two connection ports configured to communicatively connect to the infusion device and the sensor; and
wherein the device hub is configured to perform a method according to claim 1.

16. A system, comprising:
one or more processors;
a memory device comprising non-transitory computer-readable instructions that, when executed by the one or more processors, cause the one or more processors to perform a method according to claim 1.

17. A memory device comprising non-transitory computer-readable instructions that, when executed by one or more processors, cause the one or more processors to perform a method according to claim 1.

18. An infusion control device, comprising:
one or more processors;
a non-transitory machine readable memory storing instructions that, when executed by the one or more processors, cause the infusion control device to:
determine, for delivery of a fluid by an infusion pump operably connected to the infusion control device, an effective range of a measured property associated with a target of the fluid based on a predetermined profile of the fluid, an amount of the fluid, and at least one physical property of the target;
determine, while the fluid is delivered by the infusion pump, an expected trend in the measured property based on first sensor data from a sensor for a prior period of time, the predetermined profile of the fluid, the amount of the fluid being delivered by the infusion pump, and the at least one physical property of the target;
cause the infusion pump to adjust the amount of the fluid being delivered to cause the measured property to follow the expected trend;
determine, after the amount is adjusted, that a current trend in the measured property deviated from the expected trend and that the current trend will cause the measured property to fall outside the effective range within a predetermined time period; and
responsive to determining that the current trend will cause the measured property to fall outside the effective range within the predetermined period of time:
determine a delivery failure of the medication based on the current trend; and
provide an alarm of the delivery failure.
